(19) United States
(12) Patent Application Publication
(10) Pub. No.: US 2004/0118407 A1
(43) Pub. Date: Jun. 24, 2004

(54) Device for artificial respiration

(76) Inventor: Michael Kandler, Heroldsberg (DE)

Correspondence Address: ROTHWELL, FIGG, ERNST & MANBECK, P.C., 1425 K STREET, N.W., SUITE 800, WASHINGTON, DC, 20005, US

(21) Appl. No.: 10/474,690

(22) PCT Filed: Apr. 11, 2002
(86) PCT No.: PCT/EP02/04057

(30) Foreign Application Priority Data
Apr. 11, 2001 (DE) 101 18 146.9
Jul. 9, 2001 (DE) 101 33 247.5

(51) Int. Cl. A61M 16/00

(52) U.S. Cl. 128/207.14

(57) ABSTRACT

The invention relates to a device for artificial respiration of a patient by delivery of a perfluorocarbon, said device having an aerosol generator by means of which an aerosol of the perfluorocarbon is formed.

### DESCRIPTION

The invention relates to a device for artificial ventilation of a patient by delivery of a perfluorocarbon, said device having an aerosol generator by means of which an aerosol of the perfluorocarbon is formed.

Artificial ventilation is a procedure successfully used in medicine for treating patients suffering from respiratory distress, lung diseases or pulmonary failure. One cause of (acute) respiratory distress syndrome ((A)RDS), also known as hyaline membrane disease, which occurs particularly often in premature babies, is the deficit of natural lung surfactant. In adults, this syndrome can occur following lung damage, for example as a result of shock-inducing trauma, burns or infection. The endogenous surfactant has the vital function of reducing the surface tension of the natural film of liquid on the alveoli. The result of a surfactant deficit is an increase in the surface tension and a collapse of the alveoli (atelectasis) and often of the entire lung. As a consequence, the collapsed lung area cannot be ventilated and cannot take part in gas exchange. To reopen the collapsed parts, the same effort must be expended at each breath as was expended in the initial filling of the lung with air. The respiratory work of the spontaneously ventilating patient is therefore hugely increased. The subsequent burden on the body leads to physical exhaustion, and the strain on the lungs leads to worsening of the pathological state. As a result of an inadequate supply of oxygen to the body and toxic accumulation of carbon dioxide in the body, this leads, particularly in newborn babies, to a mortality rate in excess of 30%.

Such patients, both in childhood and in adulthood, are treated by means of artificial ventilation. The ventilation measures applied can take different forms (for example, continuous positive air pressure, CPAP; intermittent mandatory ventilation, IMV). By means of the artificial expansion of the lungs with the pressurized ventilation gas (for example an oxygen and air mixture), the lung which has become stiff (low compliance) as a result of disease is ventilated. As a result of the pressure and volume changes in the cycle of inhalation and exhalation, considerable shearing forces act on the walls of the airways and alveoli. These shearing forces are greater, the stiffer the lung, because more ventilation pressure then has to be applied in order to open the lung. The distension effects of the above-described procedure lead to further worsening of the disease in the form of an increase in the lung damage.

In premature and newborn babies who, in the context of RDS, have a surfactant deficit, surfactant can be administered as a medicament through the endotracheal tube (respiration tube inserted into the trachea). However, in children who, in addition to the existing surfactant deficit, have structurally immature lungs, or in patients of any age who have an abnormally high level of surfactant use as a result of shock and/or infection, administration of surfactant remains without success.

Perfluorocarbons (PFC) are polyfluorinated carbons, i.e. compounds whose carbon skeleton is at least partially perfluorinated but can also contain further halogen substituents, for example bromine. These are clear and completely chemically inert compounds which have already been used in many technical fields for some time now.

Before it was found they could be used as a surfactant substitute, they had also already been in use in medicine as a blood substitute, since oxygen, carbon dioxide and other gases are very easily soluble in them and they therefore have an oxygen and CO₂ capacity two to three times that of blood. They thus have a double action, on the one hand acting as a surfactant-like substance and on the other hand as a means of transporting gases, in particular oxygen and carbon dioxide. Together with the fact that the distensibility (compliance) of the lung is greatly improved as a result of the reduced surface tension, it is also possible to achieve oxygen uptake and CO₂ elimination in a severely diseased lung. In addition, endogenous surfactant production is induced by perfluorocarbon. There are presently no indications to suggest that very small resorbed quantities have any toxic effect. It has also been possible to rule out any pathogenic effect on lung tissue.

For some time now, perfluorocarbons have also been used for treating the lungs and in the field of artificial liquid ventilation.

WO 95/31191 discloses a method of assisting a patient's breathing, in which method a perfluorocarbon-containing liquid is introduced into the lung in order to allow the patient to breathe without any ventilation device.

DE 197 19 280 C1 discloses fluorinated alkanes of the formula R_{F}[CF₂—CH₂]R_{H} and their use as an oxygen-transporting medium and as a means of regenerating the lungs by pulmonary lavage.

EP 583 358 B1 describes the use of liquid fluorocarbons as medicaments for partial liquid ventilation (PLV). The fluorocarbon liquid is in this case used in a quantity of 0.1% to 50% of the patient's total lung capacity.

The disadvantages of total liquid ventilation (TLV), in which the lung is filled completely with a ventilation liquid, are principally that the equipment necessary for this, namely special liquid ventilation units, are very much more expensive than gas ventilators. Partial liquid ventilation (PLV) only partially solves these problems. It is true that it is possible to use a gas ventilation unit, since the lungs are filled only partially with liquid, but a large amount of ventilation liquid still has to be applied, and the liquid is distributed nonhomogeneously in the lungs on account of gravity, which is a particular disadvantage when the liquid is intended to represent a surfactant substitute. It has also been found that, in partial liquid ventilation, there are large numbers of burst alveoli in the upper part of the lung, where there is no liquid. The effect on oxygenation, ventilation and lung mechanics has been investigated in a number of animal experiments using different models of lung failure (3, 4, 5, 6). Clinical observations of the use of PLV have been made for ARDS (acute respiratory distress syndrome), meconium aspiration syndrome, congenital diaphragmatic hernia, and respiratory distress syndrome (RDS) in premature babies (7, 8, 9). PLV requires extreme care, since adjustment of the respirator parameters by the lungs filled with perfluorocarbon has different effects than in gas ventilation (11, 12). Filling the lungs and maintaining the desired level of filling requires considerable experience. Interruption of PLV leads to an immediate deterioration in the gas exchange. Tests involving incomplete filling of the FRC volume had revealed lesser efficacy of PLV (13). Because of these considerable side effects of PLV on gas exchange and on pulmonary circulation, PLV can be used only to a limited extent. To permit wider use of PAGE, techniques have to be developed which are less difficult to employ and which additionally have less potential for serious side effects.

Conventional aerosol generators are already well known. They permit aerosol formation in a wide variety of ways (single-substance jet, atomization by centrifugal force, condensation, vaporization, dispersion, ultrasound, jet nebulization, etc). The physical principles of aerosol formation are described for example in K. Dirnagel, Atemwegs-und Lungenkrankheiten [Diseases of the Airways and Lungs], volume 5, No. 1/1979, pages 22 to 27. This discusses, in particular, the two-substance jet (jet nebulizer), the ultrasonic nebulizer and the propellant gas nebulizer. In Gottschalk et al. (Atemwegs-und Lungenkrankheiten [Diseases of the Airways and Lungs], 1978, pages 378-380), a device for generating electro-anodes is described (produced by VEP Transformatoren-und Röntgenwerk “Hermann Matern”, Dresden) which can charge aerosol with 4, 1 or 0.1 kV (ion current 12, 3, or 0.3 nA). Other nebulizers are discussed in Montgomery et al. 1987, The Lancet, page 480; Matthys, 1990, Lung S., 645-652). Conventional aerosol therapy is used mainly for administering medicaments generally to nonventilated patients. It has therefore been sought to provide aerosol generators which are as small as possible and portable and which can be used by the patients themselves. Aerosol technology is therefore increasingly developing toward small metered inhalers in which individual puffs are to be inhaled by the patient, but which are not geared toward longterm ventilation. According to Dirnagel, in Atemwegs-und Lungenkrankheiten [Diseases of the Airways and Lungs], volume 12, No. 5, 1986, pages 212 to 215, the most important obstacle to methodical improvements is the fact that aerosol deposition is strongly dependent on factors which have nothing to do with the technique of aerosol generation but are determined by the behavior of the patient and by the anatomical and functional state of the patient's respiratory tract.

Aerosols are admittedly already used on artificially ventilated patients, but their use has hitherto been limited to admixing medicaments to a respiratory gas. Administration of medicaments to ventilated patients by adding the medicament to the respiratory gas is by now a standard therapy, see for example Frankel et al. Critical Care Medicine, volume 15, No. 11, page 1051, 1987; Outwater et al., AJDC, volume 142, page 512. There, an SPAG (small-particle aerosol generator) unit is connected outside the patient to a ventilation unit, and the aerosol of the medicament can be delivered to the respiratory gas via several valves. In European patent application EP 908 178 A1, which like the aforementioned EP 583 358 deals with partial liquid ventilation using perfluorocarbons, mention is admittedly made of an aerosol of fluorocarbon with a medicament, but there is no reference to administration of the respiratory liquid per se, nor to any device which would be suitable for this.

Jet nebulizers and ultrasonic nebulizers have likewise been used, but the aerosol was administered separately from the respiratory gas/respiratory liquid (Matthys, Lung (1990) Suppl.: 645-652; Montgomery et al., CHEST (1995), page 774).

Administration of an aerosol formed outside and remote from the patient and given through an endotracheal tube constitutes an important impediment to the aerosol's availability. A considerable proportion of the aerosol particles is lost through contact with the wall of the endotracheal tube, whose internal diameter is much smaller than that of the natural upper airways, i.e. the aerosol particles are converted to liquid. When administering medicaments in which the finest possible particle size is desired, this separation of the larger particles may in some circumstances even be advantageous. The disadvantage, however, is that the efficacy of the medicament is considerably reduced.

The object of the present invention was therefore to make available a device for artificial ventilation of a patient by means of a perfluorocarbon in aerosol form, which device at least partially avoids the disadvantages of the liquid ventilation methods of the prior art.

The object is achieved by a device for ventilation of a patient by delivery of a perfluorocarbon into the lungs, said device having a ventilation unit, an aerosol generator, and a tube system with a tube area connecting the tubes, communicating with the patient and comprising an endotracheal tube, said device being characterized in that the aerosol generator is arranged in the tube area conveying gas in the inhalation phase and exhalation phase, or distally from there in the patient.

Surprisingly, it has been found that artificial ventilation using a conventional perfluorocarbon in the form of an aerosol exploits the action of the perfluorocarbon both as surfactant and as gas transporter better than in conventional liquid ventilation, particularly since, in contrast to PLV, the effect of the perfluorocarbon lasts for the duration of the treatment period.

A device which permits artificial ventilation by delivering an aerosol of perfluorocarbon as ventilation medium is not previously known in the prior art. The device according to the invention thus permits a novel type of ventilation with the aid of perfluorocarbons, these being administered in the form of an aerosol.

The device according to the invention makes it possible to manage with a minimum amount of perfluorocarbon and also to rapidly distribute this amount uniformly across the entire surface of the lungs. A further advantage is that there is no accumulation of liquid, and the lung volume is not reduced by filling up with liquid. The impact on the patient is thus much less and the risk which is unavoidable in changed respiratory mechanics using liquid in the lungs is reduced. Moreover, the device according to the invention is easy to handle.

A ventilation unit suitable as a basis for the device according to the invention can be any standard ventilation unit which delivers the respiratory gas to the patient via an endotracheal tube or similar device. The Nelcor Infantstar 950 C is suitable for example.

In general, the ventilation unit used in the device according to the invention will be one comprising a tube system consisting of at least two ventilation tubes. Ventilation gas is delivered to the patient through one of these tubes and is removed from the patient through the other one. Both tubes are normally joined together at a certain point via a Y-piece. The Y-piece is generally connected to the endotracheal tube via a tube connector. This plug connection (tube connector) is provided in many ventilation units, so that endotracheal tubes of different diameter can be attached to the Y-piece depending on whether the patient concerned is an animal, a child or an adult. The tube area through which the ventilation medium can flow in both directions is referred to as the tube area which conveys gas in the inhalation phase and the exhalation phase. The other tube areas convey gas either only in the inhalation phase or only in the exhalation phase. This area conveying gas in the inhalation phase and the exhalation phase thus includes the endotracheal tube, the tube connector (if present), and the base of the Y-piece.

The aerosol generator is arranged in such a way that the aerosol is formed in the tube area conveying gas in the inhalation phase and exhalation phase, or distally from there in the patient. Referring to the illustrative embodiment of the device according to the invention shown in FIG. 1, this means that the aerosol generator is arranged in such a way that the aerosol is formed in the area labeled “P” or distally from there.

The distance which the aerosol has to travel through a tube to the patient is preferably as short as possible. It is particularly preferable for the aerosol generator to be arranged directly in the endotracheal tube.

The aerosol generator is preferably arranged in such a way that the aerosol is formed inside the patient, preferably in the area of the trachea (to ensure distribution into the lower airways) and even more preferably in the area of the lung.

The aerosol generator can also be arranged distally from the end of the endotracheal tube, that is to say protruding beyond the endotracheal tube into the patient. In this embodiment, any contact of the aerosol with tube walls is avoided, so that the aerosol can be distributed unimpeded in the trachea or the bronchi or lungs (depending on the configuration).

It is also possible to use several aerosol generators, for example one for each lung.

In this way, by forming the aerosol within the patient's body, it is possible to readily control the properties of the aerosol, for example the size of the aerosol particles, the droplet size ranges, the densities and quantities of the mist. If an aerosol is administered using a mask or a mouthpiece or tube, it can happen that fairly large quantities of the aerosolized substance are lost. However, if the aerosol is formed within the patient's body, then, as a result of the shorter distance the aerosol droplets have to travel to reach the lung surface, it is possible to better control the size, distribution and quantity. This avoids condensation of the aerosol in the ventilation tube and thus ensures that the quantity of the aerosol which is delivered to the patient is not reduced.

The aerosol generator according to the invention is a device from which the aerosol emerges at its end. It can consist of a jet nozzle, a tube, a catheter or a similar arrangement.

If a catheter is used, the catheter tip can be arranged in the inside of the tube area conveying gas in the inhalation phase and exhalation phase, preferably in the endotracheal tube. The catheter tip particularly preferably lies at the end of the endotracheal tube, or it protrudes slightly beyond it into the patient.

It is also possible, and for the present invention it is preferable, to design the endotracheal tube in such a way that it itself assumes the function of an aerosol generator. For example, the walls of the endotracheal tube can be provided with cavities, in particular ducts, which have openings either on the inner wall of the endotracheal tube or in the outer wall or in both. These openings can then function as a nozzle, the aerosol being formed at the openings.

The way in which the aerosol is generated is not critical to the present invention, and it can be done by any suitable method, for example by means of a single-substance jet, two-substance jet, atomization by centrifugal force, condensation, vaporization, propellant gas, dispersion, ultrasound, jet nebulization, etc. (see above). Ultrasonic nebulization and jet nebulization are particularly preferred. In some circumstances, combinations may also be advantageous. Each type of aerosol generation mentioned can be combined, if desired, with a catheter.

The aerosol is preferably generated by mixing the perfluorocarbon with a suitable ventilation medium. The ventilation medium is preferably a mixture of air and oxygen, but it is also possible to use other suitable mixtures, or air or oxygen individually.

The device particularly preferably has, as aerosol generator, a nebulization catheter. A suitable catheter is, for example, the Trudell nebulization catheter from the company Trudell Medical Group.

This technique consists of a very small catheter (external diameter 0.1 to 2 mm, depending on type) and uses a nebulization technique involving pressure liquid/pressure gas in order to form an aerosol at the outermost end of the catheter. It consists of a single extrusion with a plurality of gas and liquid capillaries. These capillaries converge and end in tiny openings at the outermost end of the catheter. Gas and liquid flow through the respective capillaries and emerge through the openings. The close contact between the gas and the liquid results in extremely efficient nebulization with low gas flow velocities, in the range of about 1 ml/min to 0.05 l/min. To operate this system, any gas mixture with a pressure of over 50 psi can be used. For the present invention, a mixture of oxygen and air is particularly suitable, although other customary ventilation gases known to the skilled person can also be used. Controlled delivery of gas and liquid in a separate device is also possible. The liquid can be introduced in coordination with the inhalation phase of a ventilator or continuously injected by hand or delivered by machine. The catheter delivers over 95% of the original liquid volume into the lung.

The particle size of the aerosol droplets can be set in advance or can be chosen by regulating the behavior of solution/liquid. This can also be done by selecting a suitable catheter type. In this way, aerosols with mean aerosol particle diameters of about 5 μm and over can be obtained, depending on the desired output and gas flow.

The perfluorocarbons used can be all known perfluorocarbons which are also suitable for partial liquid ventilation. Perfluorocarbons are understood as nontoxic, preferably liquid, fluorinated carbon compounds which are suitable for gas exchange and are thus suited for ventilation of human or animal patients. Instead of being substituted with fluorine, they can also be substituted with other halogens. Perflubron (perfluorooctyl bromide, PFOB, C₈F₁₇Br) from the company Alliance, perfluorodecaline (C₁₀F₁₈) from F2-Chemicals GB, FC 77 (C₈F₁₇O), FC5080 (C₈F₁₈), FC43 (C₁₂Fₓ), FC 3280, FC3283 from 3M, and the mixture RM 101 from Miteni, are particularly suitable. As regards toxicity, this depends on the purity with regard to fluorine radicals which can be later cleaved off or hydrogen fluoride. Other suitable perfluorocarbons are mentioned in EP 908 178 A1.

The quantity of perfluorocarbon in aerosol form advantageously used depends on the properties of the particular perfluorocarbon or perfluorocarbon mixture, in particular the vapor pressure and viscosity. It is important that the lung surface is reached by the aerosol in such a way that a sufficient gas exchange can take place and that there is sufficient lining of the lung surface with perfluorocarbon. The aim is for the lung surface to be wetted, but large accumulations of liquid are unnecessary and are not desired.

A further aspect of the present invention is the use of an aerosol generator, for generating an aerosol of a perfluorocarbon suitable for ventilation of a patient, in combination with a ventilation device. The aerosol generator used can be any of the aforementioned aerosol generators.

A further aspect of the present invention is the use of perfluorocarbons in the form of aerosols as means for artificial ventilation of a patient. As has already been mentioned in the introduction, the conventional uses of perfluorocarbons, namely in TLV and PLV, have considerable disadvantages. Thus, a number of indications for the conventional means of artificial ventilation could be treated only with difficulty. Surprisingly, treatment is very much more effective using an aerosol of a perfluorocarbon. Perfluorocarbons in the form of aerosols are thus suitable as means for treatment of ARDS and RDS (surfactant deficit), lung damage, and pulmonary hypoplasia, which, for example, may have its origin in the immature state of the lungs or may be localized, for example in the case of diaphragmatic hernias.

A further aspect of the present invention is therefore also a method for artificial ventilation of a patient by delivery of a perfluorocarbon to the lungs, the perfluorocarbon being converted to aerosol form in a tube area, which conveys gas in the inhalation phase and exhalation phase, comprises an endotracheal tube, and communicates with the patient, and being delivered to the lungs.

In a further embodiment of the present invention, the perfluorocarbons are used in combination with medicaments.

Thus, the perfluorocarbon delivered as aerosol also fulfills the function of transporting the medicament. In particular, it is possible to distribute the medicament into lung areas which are difficult to access or which have previously been closed. To this end, the medicament is incorporated into the perfluorocarbon by a suitable method, for example emulsification, micellation, or by additional mediator substances. The medicaments can also be packed in vehicles and admixed to the perflurocarbon, for example packed in liposomes or viral vehicles, for example vectors. The appropriate type of admixing and packing of the medicaments can be applied by the skilled person depending on the substances used. It is also possible to use the medicament in the form of PulmoSpheres™ (obtainable from Alliance) in combination with the perfluorocarbon.

All medicaments that can be taken up via the lungs are suitable in principle. Specific treatments of the lungs can be carried out by appropriate incorporation of medicaments into the perfluorocarbon used. Preferred areas of application are the indications already mentioned herein, and also indications in which, for example, vasodilating drugs (adrenomedullin, prostacyclin), anti-inflammatory drugs (e.g. corticosteroids), and cytostatic drugs (e.g. cisplatin, 5-fluorouracil) are used.

It is also possible to effectively use the aerosol as a transporter in gene transfer, including the transfer of RNA (and RNAi), for example via vectors or liposomes.

Since the particular medicament is introduced together with the perfluorocarbon aerosol into the lungs in a targeted manner, it is possible to ensure a more exact metering and better uptake of the medicament than would be the case when adding medicaments to the ventilation medium in TLV or PLV. Thus, the present invention also permits a more targeted and more effective treatment of serious diseases, for example cystic fibrosis.

The following figures and examples are intended to illustrate in more detail a number of embodiments of the invention.

### FIGURES

FIG. 1 shows a device according to the invention, consisting of a conventional ventilation unit (1) which is connected by an endotracheal tube (2) to the patient (4) and an aerosol generator (3). Two tubes (5, 6) through which respiratory gas can flow are connected to the ventilation unit. Gas flows through one tube (5) always in the direction of the patient (inhalation), and through the other tube (6) it always flows away from the patient (exhalation). The two tubes (5, 6) come together at a Y-piece (7) and are thus connected to the endotracheal tube via a tube connector (8). The tube area labeled P is the area which conveys gas in the inhalation phase and the exhalation phase. The aerosol generator is arranged in such a way that the aerosol is formed in the area P, which conveys gas both in the inhalation phase and the exhalation phase, specifically in this case at the end of the endotracheal tube.

FIG. 2 shows a Trudell catheter (9) which is inserted into an endotracheal tube (22). Here, the aerosol (10) is formed at the catheter tip in the patient's body.

FIG. 3 shows the arterial oxygen partial pressure (PaO₂) as mean±SEM before and after induction of lung failure, during therapy with PFC aerosol, FRC-PLV, LV-PLV, control, and during the observation phase after treatment in surfactant-depleted newborn piglets.

FIG. 4 shows the PaCO₂ as mean±SEM before and after induction of lung failure, during therapy with PFC aerosol, FRC-PLV, LV-PLV, control, and during the observation phase after treatment in surfactant-depleted newborn piglets.

FIG. 5 shows the terminal compliance C20/c as mean±SEM for determining the terminal dynamic compliance and lung overdistension. High C20/c values point to a high terminal dynamic compliance and to a reduction of the lung overdistension. Obtained before and after induction of lung failure, during therapy with PFC aerosol, FRC-PLV, LV-PLV, control, and during the observation phase after treatment in surfactant-depleted newborn piglets.

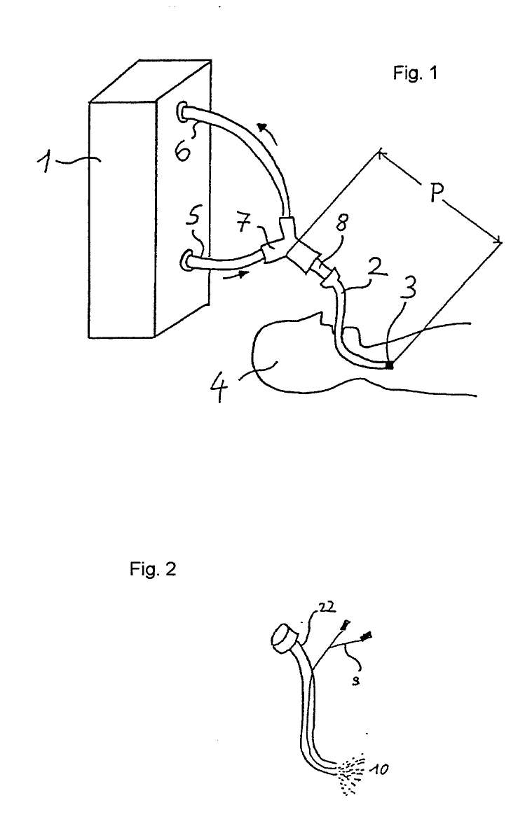

With this device it was possible to achieve a particle size spectrum of from 5 to 20 μm.

### EXAMPLE 1

Sustained Improvement of Gas Exchange and Lung Mechanics through Ventilation with Aerosolized Perfluorocarbon.

The effect of aerosolized perfluorocarbon on oxygenation, ventilation and lung mechanics was investigated in surfactant-depleted piglets with resultant ARDS. At present, we know of no study which has described the use of aerosolized PFC. Successful treatment was carried out with FC77 (see below), perfluorooctyl bromide (C₈F₁₇Br), FC43, and perfluorodecalin (results not shown).

Material and Methods.

The study was approved by the competent authority for the protection of animals. Twenty newborn piglets with a bodyweight of between 3.5 and 4.3 kg, and a maximum of ten days old, were selected directly for the investigation. The animals were sedated with azaperone (1 mg/kg bodyweight) and Dormicum® (midazolam 0.5 mg/kg bodyweight) i.m. and fitted with a peripheral indwelling venous cannula. Anesthesia was induced with a bolus injection of 5 mg/kg Ketanest S® (ketamine), 1.0 mg/kg Dormicum® and 2.5 μg/kg fentanyl, followed by a continuous infusion of 1.5 mg/kg/h midazolam, 0.01 mg/kg/h fentanyl, and 15 mg/kg/h Ketamin S. The animals were tracheotomized, and an endotracheal tube (Mallinckrodt®, i.d. 4.0 mm) was placed with its distal end 3.5 cm above the bifurcation of the trachea.

The endotracheal pressure was measured using a 5 Ch catheter 10 mm above the bifurcation. The position of both was checked by rigid bronchoscopy. Following insertion of the endotracheal tube, mechanical ventilation was started. The animals were relaxed with 0.2 mg/kg bodyweight Norcuron® (vecuronium), followed by a continuous infusion of 0.2 mg/kg/bodyweight/h of vecuronium. A repeat dose of fentanyl (5 μg/kg bodyweight) was also injected intravenously. A 4.5 Ch lock (Cook®, Germany) was inserted surgically into the right jugular vein. A 4 Ch thermodilution catheter (Arrow®, Erding, Germany) was introduced though this in order to measure the pulmonary artery pressure and cardiac output. A 20-gauge cannula (Arrow®) was introduced surgically into the right femoral artery, and a Paratrend 7 fluorescence sensor was introduced for online measurement of the blood gases. The arterial blood gas analysis was carried out at intervals of 15 minutes during treatment, and at intervals of thirty minutes in the observation phase. (ABL 330, Radiometer Copenhagen, Denmark). The tidal volumes were measured with a hot-wire anemometer (MIM® GmbH, Krugzell) and plotted with the neonatal respiration monitor Florian® NRM-200 (MIM®). To identify lung overdistension, the C20/c (20% terminal dyn. compliance/dyn. compliance) (18) was measured. Conventional ventilation (intermittent mandatory ventilation, IMV) was carried out with the neonate ventilation unit Infant Star 950 (Mallinckrodt, Hennef, Germany). The respiratory gas was warmed to 39° C. and humidified. (MR 700, Fischer & Paykel, Welzheim, Germany). The respiratory rate was 50 breaths per minute, and a peak pressure (PIP) of 32 cm H₂O and a positive end-expiratory pressure (PEEP) of 8 cm H₂O were set.

Lung failure was induced by repeated bronchoalveolar lavage with physiological saline solution (0.9%), using a lavage volume of in each case 30 ml/kg. The criterion for adequate lung damage was a PaO₂ of below 80 mmHg for a duration of one hour. Once lung failure had been fully induced, the animals were allocated at random to one of the following treatment groups:

1. PFC aerosol, 2. FRC-PLV, 3. low-volume (LV)-PLV, 4. control (IMV). The ventilation parameters were not changed, so as to ensure comparability of the groups. The PFC aerosol group received 10 ml/kg/h of FC77® (C₈F₁₈ and C₈F₁₆O, density 1.78 g/cm³, 3M®, Neuss, Germany) (20, 31) through an aerosol catheter (oxygen jet aerosol generator, Trudell Medical Inc.™, Toronto, Canada), (particle diameter 5-8 μm). The catheter consists of a bundle of capillaries which guide gas and liquid to the tip of the catheter and, as a result of the convergent shape at the tip, form an aerosol there. Highly effective nebulization was achieved with a gas flow of 0.05 liter of oxygen per minute. The LV-PLV group received 10 ml/kg/h of FC77® in liquid form through an endotracheal tube. The lungs of the FRC-PLV group were filled with 30 ml/kg of FC77® for a period of 30 minutes, followed by a substitution quantity of 20 ml/kg/min in order to compensate for the evaporation loss. The control group was ventilated with IMV. After two hours, the specific form of ventilation was ended, and the animals were monitored for a period of six hours under IMV.

Data analysis and statistics: Values as mean±SEM. After the data had been examined for the presence of a Gaussian distribution, the two-way ANOVA was used to check for the presence of a significant difference between the groups. In the event of a significant difference, the Bonferroni post-hoc test was also applied. A p-value of below 0.05 was assessed as significant.

Results

Arterial Oxygen Partial Pressure (PaO₂):

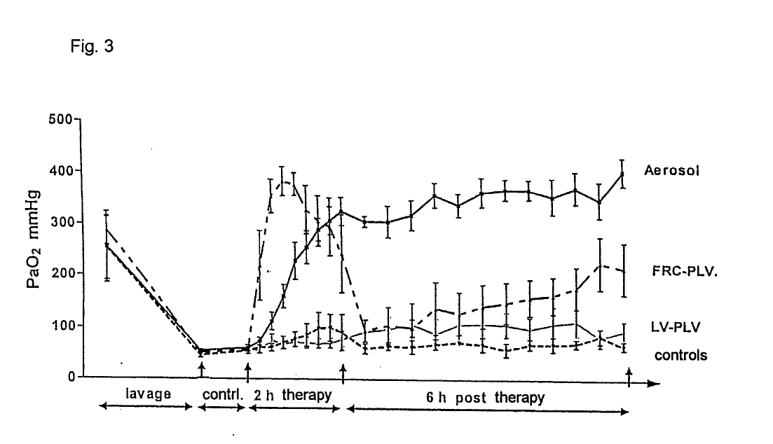

Treatment with aerosolized perfluorocarbon increased the PaO₂ significantly compared to the untreated control group (p<0.001) and compared to the LV-PLV group (p<0.001). The increase in PaO₂ in the animals treated with PFC aerosol was flatter than in the FRC-PLV group (FIG. 3). After completion of the PFC therapy, there was a sustained increase in the PaO₂ in the PFC aerosol group, but not in the FRC-PLV group. Six hours after the end of therapy, the piglets treated with PFC aerosol had significantly higher PaO₂ values than all the other groups. (p<0.01): PFC aerosol: 406.4±26.9 mmHg, FRC-PLV: 217.3±50.5 mmHg, LV-PLV: 96.3±18.9 mmHg, control group: 67.6±8.4 mmHg; p<0.001.

Oxygenation Index:

The oxygenation index (OI) was calculated using the following formula:

([MAP(cmH₂O)×FiO₂/PaO₂ (mmHg)]×100) (16).

The OI increases with increased mean airway pressure (MAP) and increased inspiratory oxygen concentration (FiO₂), and the OI falls with increasing PaO₂. Inversely proportional to the increase in PaO₂, the OI fell from 29.9±3.4 to 17.1±3.8 during the first 30 minutes of the PFC aerosol therapy. The fall in the OI in the first 30 minutes was markedly quicker and more pronounced in the FRC-PLV group (from 31.3±1.5 to 5.1±0.6), but after two hours of treatment the OI in both groups, PFC aerosol and FRC-PLV, was significantly lower than in the LV-PLV group and the control group (p<0.001). The increase in the OI achieved by PFC aerosol treatment compared to the control group was still unchanged six hours after the end of therapy (p<0.001). In the FRC-PLV group, by contrast, there was a rapid deterioration in the OI after the end of the PFC instillation, with an increase in the OI from 7.7±2.5 to 21.2±13.3 within 15 minutes (Table 1).

Carbon Dioxide Elimination:

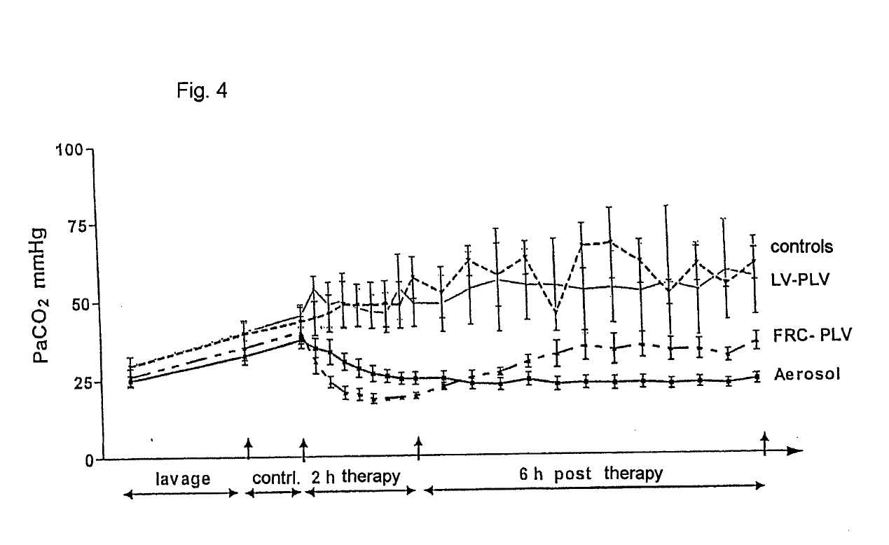

Sixty minutes after the start of therapy, the PFC aerosol treatment was associated with significantly lower PaCO₂ values than the animals in the control group (p<0.01) and those treated with LV-PLV (p<0.01) (FIG. 4). This effect was sustained for six hours after the end of the aerosol therapy (p<0.01). After thirty minutes of treatment with FRC-PLV, the PaCO₂ in this group was significantly lower than that of the control group and the LV-PLV group (p<0.01). Eight hours after the start of perfluorocarbon treatment, there was only a statistically nonsignificant difference in PaCO₂ in the PFC aerosol group and FRC-PLV group (24.2±1.7 mmHg versus 35.9±2.8 mmHg). The lowest PaCO₂ was measured in the PFC aerosol group: 24.2±1.7 mmHg, FRC-PLV: 35.9±2.8 mmHg, LV-PLV: 56.7±12.4 mmHg, control group: 60.6±5.1 mmHg; p<0.01).

Ventilatory Efficacy Index:

In order to describe the ventilation independently of the applied ventilation parameters, the ventilatory efficacy index (VEI) was calculated:

(3800/(PIP-PEEP(cmH₂O))×respiratory frequency (cpm)×PaCO₂) (17).

Sixty minutes after the start of therapy, the VEI in the group treated with PFC aerosol was significantly higher than in the control group (0.116±0.011 versus 0.071±0.011; p<0.05). The most rapid increase in the ventilatory efficacy index was observed in the FRC-PLV group. After thirty minutes, the VEI in the FRC-PLV group was significantly higher than the VEI of the control group (0.099±0.011 versus 0.071±0.011; p<0.05). After the PFC delivery ended, the VEI in the FRC-PLV group fell to values of the initial level before start of therapy, but not in the PFC aerosol group (Table 1).

Lung Function:

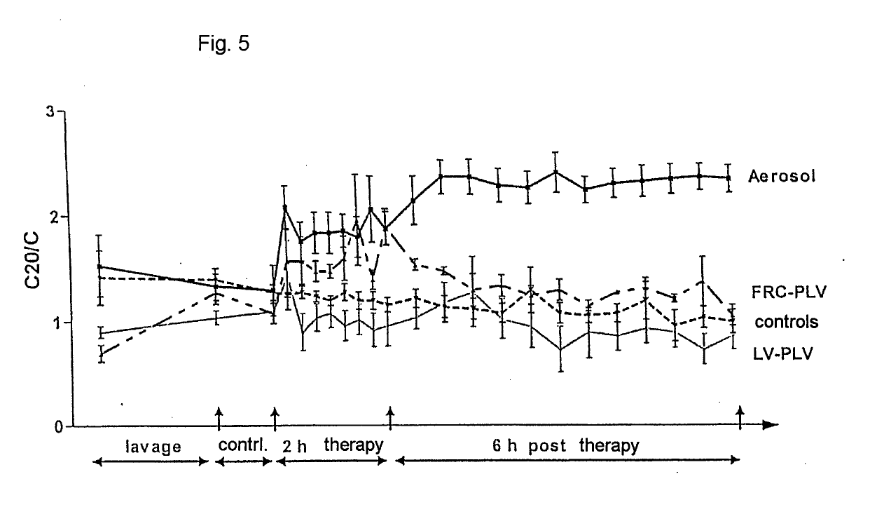

The dynamic compliance improved within 15 minutes in the PFC aerosol group and the FRC-PLV group. High C20/c values point to a high terminal dynamic compliance and to a reduction in lung overdistension (17). After 120 minutes of PFC aerosol treatment, the C20/c was significantly (p<0.01) higher than in the control group and the LV-PLV group (FIG. 5). Although, after two hours of treatment, the FRC-PLV group showed significantly higher C20/c values than the control group and the LV-PLV group (p<0.001), this positive effect ended immediately upon completion of therapy. In the following observation period, there was no longer any significant difference in relation to the control group. By contrast, there was a sustained increase in C20/c in the PFC aerosol group after therapy, by twice the values measured in the control group and the LV-PLV group (p<0.01), and the C20/c values in the PFC aerosol group were significantly higher than in the FRC-PLV group (p<0.001). The terminal compliance, C20/c, was highest in the PFC aerosol group (p<0.001).

Circulatory Parameters:

There was no significant difference between the groups in terms of cardiac output, central venous pressure and body temperature.

Safety Measures:

The absence of an air leak and the position of the thermodilution catheter were checked by radioscopy. The endotracheal pressure was identical in all the groups. During the observation phase, one of the animals in the LV-PLV group had tension pneumothorax.

Ventilation with aerosolized perfluorocarbon improved the pulmonary gas exchange and lung mechanics just as much as did partial liquid ventilation, and the effect lasted longer.

The examination of the lung samples for the presence of any inflammatory reaction caused by ventilation trauma showed that ventilation with PFC aerosol and with FRC-PLV leads to a significantly lower interleukin 1-beta and interleukin-8 gene expression than does ventilation with LV-PLV or conventional ventilation (K. von der Hardt, M. Kandler et al., submitted for publication). One reason could be the improvement in pulmonary gas exchange without the pressure-induced alveolar overdistension. The reduced barotrauma and volutrauma with less shearing force leads to a reduction in the inflammation cascade of ARDS and thus to a reduction in the self-perpetuating disease process (26, 27, 28, 29, 30).

In summary, the present investigation shows that treatment with aerosolized perfluorocarbon represents a novel, effective, safe and easy-to-use method of perfluorocarbon administration for ventilation.

TABLE 1
Oxygenation Index (OI) and Ventilatory Efficacy Index (VEI)

| | PFC aerosol | | FRC-PLV | | LV-PLV | | Control |
|---|---|---|---|---|---|---|---|
| OI | | | | | | | |
| before therapy | 29.8 ± | 3.4 | 31.3 ± | 1.4 | 32.2 ± | 1.7 | 33.6 ± 4.4 |
| 1 h therapy | 8.3 ± | 1.2**††† | 4.7 ± | 0.3*†† | 26.7 ± | 3.2 | 25.5 ± 4.5 |
| 2 h therapy | 5.4 ± | 0.4***†††‡‡ | 21.2 ± | 13.3 | 26.7 ± | 4.8 | 26.7 ± 5.3 |
| 6 h post therapy | 4.4 ± | 0.3***† | 11.3 ± | 3.5 | 20.1 ± | 4.7 | 27.7 ± 3.1 |
| VEI | | | | | | | |
| before therapy | 0.08 ± | 0.003 | 0.08 ± | 0.01 | 0.07 ± | 0.006 | 0.08 ± 0.01 |
| 1 h therapy | 0.11 ± | 0.01*** | 0.16 ± | 0.01***††† | 0.07 ± | 0.09 | 0.07 ± 0.01 |
| 2 h therapy | 0.13 ± | 0.01*** | 0.16 ± | 0.008***††† | 0.07 ± | 0.01 | 0.06 ± 0.008 |
| 6 h post therapy | 0.13 ± | 0.01***†‡ | 0.09 ± | 0.006 | 0.06 ± | 0.01 | 0.05 ± 0.004 |

***p < 0.001,
**p < 0.01,
*p < 0.05 versus con†rol
†††p < 0.001,
††p < 0.01,
†p < 0.05 versus LV-PLV
‡‡‡p < 0.001,
‡‡p < 0.01,
‡p < 0.05 versus FRC-PLV

### LITERATURE

1. Fuhrmann B P, Paczan P R, and DeFrancis M. 1991. Perfluorocarbon-associated gas exchange. Crit. Care Med. 19: 712-722.

2. Greespan J S, Wolfson M R, Rubenstein S D, and Shaffer T H. 1990. Liquid ventilation of human preterm neonates. J. Pediatr. 117: 106-111.

3. Greespan J S, Wolfson M R, Rubenstein S D, Antunes N E, Kechner P, and Shaffer T H. 1995. Partial liquid ventilation trials: management implications of serial pulmonary mechanics. Pediatr. Res. 37: 207A.

4. Kirmse M, Fujino Y, Hess D, and Kacmarek R M. 1998. Positive end-expiratory pressure improves gas exchange and pulmonary mechanics during partial liquid ventilation. Am. J. Resp. Crit. Care Med. 158: 1550-1565.

5. Papo M C, Paczan P R, Fuhrman B P, Steinhorn D M, Hernan L J, Leach C L, Holm B A, Fisher J E, and Kahn B A. 1996. Perfluorocarbon-associated gas exchange improves oxygenation, lung mechanics, and survival in a model of adult respiratory distress syndrome. Crit Care Med. 24 (3): 466-474.

6. Parent A C, Overbeck M C, and Hirschl R B. 1997. Oxygen dynamics during partial liquid ventilation in a sheep model of severe respiratory failure. Surgery 121 (3): 320-327.

7. Leach C L, Greespan J S, Rubenstein S D, Shaffer T H, Wolfson M R, and Jackson J C. 1996. Partial liquid ventilation with perflubron in infants with severe respiratory distress syndrome. N. Engl. J. Med. 335 (11) 761-767.

8. Pranikow T, Gauger P G, and Hirschl R B. 1996. Partial liquid ventilation in newborn patients with congenital diaphragmatic hernia. J. Pediatr. Surg. 31(5): 613-614.

9. Wilcox D T, Glick P L, Karamanoukian H L, Morin F C, Fuhrmann B P, and Leach C L. 1997. Partial liquid ventilation and nitric oxide in congenital diaphragmatic hernia. J. Pediatr. Surg. 32(4): 1211-1215.

10. Shaffer T H, Tran N, Bhutani V K, and Sivieri E. 1993. Cardiopulmonary function in very preterm lambs during liquid ventilation. Pediatr. Res. 17: 640-644.

11. Tütünc\ddot{u}A S, Faithfull S, and Lachmann B. 1993. A comparison of ventilatory support with intratracheal perfluorocarbon administration and conventional mechanical ventilation in animals with acute respiratory failure. Am. Rev. Resp. Dis. 144: 745-792.

12. Merz U, Kellinghaus M, Hausler M, Pakrawan N, Klosterhalfen B, and Hörnchen H. 2000. Partial liquid ventilation with surfactant: effects on gas exchange and lung pathology in surfactant-depleted piglets. Intensive Care Med. 26 (1): 109-116.

13. Olschewski H, Ghofrani H A, Schmehl T, Winkler J, Wilkens H, Hoper M M, Behr J, Kleber F X, and Seeger W. 2000. Inhaled iloprost to treat severe pulmonary hypertension. An uncontrolled trial. German PPH Study Group. Ann. Intern. Med. 21; 132(6): 435-443.

14. Lutz C, Carney D, Finck C, Picone A, Gatto L A, Paskanik A, Langenback E, and Nieman G. 1998. Aerosolized surfactant improves pulmonary function in endotoxin-induced lung injury. Am. J. Respir. Crit. Care Med. 158(3): 840-845.

15. Bartlet R H, Toomasian J, Roloff D, Gazzaniga A B, Corvin H G, Rucker R. 1986. Extracorporeal membrane oxygenation (ECMO) in neonatal respiratory failure. Annals of Surgery 204: 236-245.

16. Notter R H, Egan E A, Kwong M S. 1985. Lung surfactant replacement in premature lambs with extracted lipids from bovine lung lavage: effects of dose, dispersion technique and gestational age. Pediatr Res 19: 569-577.

17. Fisher J B, Mammel M C, Coleman J M, Bing D R, Boros S J. 1988. Identifying lung overdistension during mechanical ventilation by using volume pressure loops. Pediatr Pulmonol 5(1): 10-14.

18. Wolfson M R, Kechner N E, Roache R F, DeChadarevian J-P, Friss H E, Rubenstein S D, Shaffer T H. 1998. Perfluorochemical rescue after surfactant treatment: effect of perflubron dose and ventilatory frequency. J Appl Physiol 84(2): 624-640.

19. Lachmann B, Robertson B, and Vogel J. 1980. In vivo lung lavage as an experimental model of the respiratory distress syndrome. Acta Anesth. Scand. 24: 231-236.

20. Hirschl R B, Tooley R, Parent A, Johnson K, and Bartlet R H. 1996. Evaluation of gas exchange, pulmonary compliance, and lung injury during total and partial liquid ventilation in the acute respiratory distress syndrome. Crit. Care Med. 24(6): 1001-1004.

21. Houmes R J M, Verbrugge S J C, Hendrik E R, and Lachmann B. Hemodynamic effects of partial liquid ventilation with perfluorocarbon in acute lung injury. Intensive Care Med. 21: 966-972.

22. Marraro G, Bonati M, Ferrari A, Barzaghi M M, Pagni C, Bortolotti A, Galbiati A, Luchetti M, and Corce A. 1998. Perfluorocarbon broncho-alveolar lavage and liquid ventilation versus saline broncho-alveolar lavage in adult guinea pig experimental model of meconium inhalation. Intensive Care Med. 24: 501-508.

23. Bleyl J U, Ragaller M, Tscho U, Regner M, Kanzow M, Hubler M, Rasche S, and Albrecht M. 1999. Vaporized perfluorocarbon improves oxygenation and pulmonary function in an ovine model of acute respiratory distress syndrome. Anesthesiology 91(2): 462-469.

24. Tooley R, Hirschl R B, Parent A, and Bartlet R H. 1996. Total liquid ventilation with perfluorocarbons increases pulmonary end-expiratory volume and compliance in the setting of lung atelectasis. Crit. Care Med. 24(2): 264-273.

25. Quintel M, Heine M, Hirschl R B, Tillmanns R, and Wessendorf V. 1998. Effects of partial liquid ventilation on lung injury in a model of acute respiratory failure: a histologic and morphometric analysis. Crit. Care Med. 26: 833-843.

26. Rotta A T, Gunnarson B, Hernan L J, Fuhrman B P, and Steinhorn D M. 1999. Partial liquid ventilation influences pulmonary histopathology in an animal model of acute lung injury. J. Crit. Care 14: 84-92.

27. Groneck P, and Speer C P. Pulmonary inflammation in the pathogenesis of broncho-pulmonary dysplasia. Pediatr. Pulmonol. Suppl. 16: 29-30.

28. Croce M A, Fabian T C, Patton J H Jr, Melton S M, Moore M, and Trenthem L L. 1998. Partial liquid ventilation decreases the inflammatory response in the alveolar environment of trauma patients. J. Trauma 45: 273-280.

29. Brunch L A, Flint A, and Hirschl R B. 1997. Pulmonary pathology of patients treated with partial liquid ventilation. Mod. Pathol. 10(5): 463-464.

30. Demirakca S, Dötsch J, Knothe C., Maagsam J, Reiter H L, Bauer J, and Kuehl P G. 1996. Inhaled nitric oxide in neonatal and pediatric acute respiratory distress syndrome: dose response, prolonged inhalation and weaning. Crit. Care Med. 24: 1913-1919.

31. Shaffer T H, Greenspan J S, Wolfson M R. 1995. Liquid ventilation. Therapies for neonatal respiratory failure. A physiological approach. Eds. Bayton, Carlo, Jabe. Cambridge University Press: 279-301.

32. MacIntyre N, Andjuval S, Baran G. 1996. Aerosol deposition from an Intra-Airway Aerosol. The European Respirator Journal: Volume 9, Supplement 23, PO396.

1. Use of an aerosol delivery device in a patient, said device containing a ventilation unit, an aerosol generator and a tube system having a tube area connecting the tubes, communicating with the patient, conveying gas in the inhalation phase and exhalation phase and comprising an endotracheal tube, and said aerosol generator being arranged in the tube area conveying gas in the inhalation phase and exhalation phase or distally from there in the patient, for producing a perhalocarbon aerosol suitable for inhalation for the ventilation of a patient.

2. The use according to claim 1,
characterized in that
the aerosol generator is arranged in the endotracheal tube or distally from there in the patient.

3. The use according to any of claims 1 or 2,
characterized in that
the wall of the endotracheal tube is provided with cavities having openings into the inner and/or outer wall of the tube, and the aerosol is generated at said openings.

4. The use according to any of claims 1 to 3,
characterized in that
the aerosol generator has a catheter.

5. The use according to any of the preceding claims,
characterized in that
the aerosol generator is suitable for generating aerosol by means of jet nebulization, atomization by centrifugal force, condensation, vaporization, propellant gas, dispersion or ultrasound.

6. The use according to any of the preceding claims,
characterized in that
a medicament is admixed to the perfluorocarbon.

7. The use of perfluorocarbons in the form of aerosols for artificial ventilation of a patient.

8. The use of perfluorocarbons in the form of aerosols for treating ARDS or RDS.

9. The use of perfluorocarbons in the form of aerosols for treating lung hypoplasia.

10. The use of perfluorocarbons in the form of aerosols for the treatment of pneumonia.

11. The use according to any of claims 7 to 10,
characterized in that
the perfluorocarbon is used in combination with a medicament.

12. A method of ventilating a patient by delivery of a perfluorocarbon into the lungs,
characterized in that
said perfluorocarbon is aerosolized in a tube area conveying gas in the inhalation phase and exhalation phase, comprising an endotracheal tube and conmunicating with the patient, and delivered to the lungs.

13. The method according to claim 12,
characterized in that a medicament is admixed to the perfluorocarbon.
